(19) United States
(12) Patent Application Publication
(10) Pub. No.: US 2023/0274191 A1
(43) Pub. Date: Aug. 31, 2023

(54) METHODS AND SYSTEMS FOR CONNECTING FOOD INTERESTS WITH FOOD PROVIDERS

(71) Applicant: KPN INNOVATIONS, LLC., Lakewood, CO (US)

(72) Inventor: Kenneth Neumann, Lakewood, CO (US)

(73) Assignee: KPN INNOVATIONS, LLC., LAKEWOOD, CO (US)

(21) Appl. No.: 18/195,067

(22) Filed: May 9, 2023

Related U.S. Application Data
(63) Continuation-in-part of application No. 16/890,796, filed on Jun. 2, 2020, now Pat. No. 11,651,413.

(51) Int. Cl. G06N 20/00; G06Q 30/0251

(52) U.S. Cl. CPC G06N 20/00; G06Q 30/0255

(57) ABSTRACT

A system for, and method of, generating textual outputs based on descriptor classifications, including a computing device configured to receive a specified location and a nourishment intake theme from a remote device, generate, as a function of a group machine-learning model, a user group theme for the first remote device, identify at least a food provider located within the specified location, determine, as a function of an descriptor machine-learning model, a descriptor classification for the first remote device and generate at least an user interface element at the first remote device as a function of the descriptor classification.

### CROSS-REFERENCE TO RELATED APPLICATIONS

This application is a continuation-in-part of Non-provisional application Ser. No. 16/890,796 filed on Jun. 2, 2020 and entitled “METHODS AND SYSTEMS FOR CONNECTING FOOD INTERESTS WITH FOOD PROVIDERS,” the entirety of which is incorporated herein by reference.

### FIELD OF THE INVENTION

The present invention generally relates to the field of artificial intelligence. In particular, the present invention is directed to methods and systems for connecting food interests with food providers.

### BACKGROUND

Locating food providers that suggest food options that align with one's food interests can be difficult. Frequently, one can be tempted by foods that may not be ideal for one's body. Knowing which foods may align with one's food interests can be challenging given the multiplicity of channels and insurmountable options available.

### SUMMARY OF THE DISCLOSURE

In an aspect, system for generating textual outputs based on descriptor classifications, the system including a computing device, the computing device designed and configured to: receive an input from a first remote device of a plurality of remote devices, wherein the input comprises a specified location and a nourishment intake theme, generate, as a function of a group machine-learning model, a user group theme for the first remote device, wherein the group machine-learning model receives the nourishment intake theme and the specified location as an input, and outputs the user group theme, identify at least a food provider located within the specified location, determine, as a function of a descriptor machine-learning model, a descriptor classification for the first remote device, wherein the descriptor machine-learning model receives the at least a food provider and the user group theme as input and outputs the advertisement theme, generate at least an advertising material for the first remote device as a function of the advertisement.

In an aspect, method of generating textual outputs based on descriptor classifications, the method including receiving, by a computing device, an input from a first remote device of a plurality of remote devices, wherein the input comprises a specified location and a nourishment intake theme, generating, by the computing device, a user group theme for the first remote device as a function of a group machine-learning model, wherein the group machine-learning model receives the nourishment intake theme and the specified location as an input, and outputs the user group theme, identifying, by the computing device, at least a food providers located within the specified location, determining, by the computing device, a descriptor classification for the first remote device as a function of an descriptor machine-learning model, wherein the descriptor machine-learning model receives the at least a food provider and the user group theme as input and outputs the descriptor classification and generating, by the computing device, at least a user interface element at the first remote device as a function of the descriptor classification.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for connecting food interests with food providers;

FIG. 2 is a block diagram illustrating an exemplary embodiment of a system for generating advertisements based on user groups;

FIG. 3 is a block diagram illustrating an exemplary embodiment of a provider database;

FIG. 4 is a diagrammatic representation of comparing a remote device with a food provider;

FIG. 5 is a process flow diagram illustrating an exemplary embodiment of a method of connecting food interests with food providers;

FIG. 6 is a process flow diagram illustrating an exemplary embodiment of a method of generating advertisements based on user groups; and

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for connecting food interests with food providers. In an embodiment, a computing device receives a plurality of inputs containing food interests data. A first machine-learning process is utilized to calculate a nourishment intake theme for each of the plurality of inputs. A computing device generates as a function of a second machine-learning process a nourishment provider theme 144 for each of a plurality of food providers. A computing device compares a nourishment intake theme with a food provider that offers nourishment provisions that compare the nourishment intake theme.

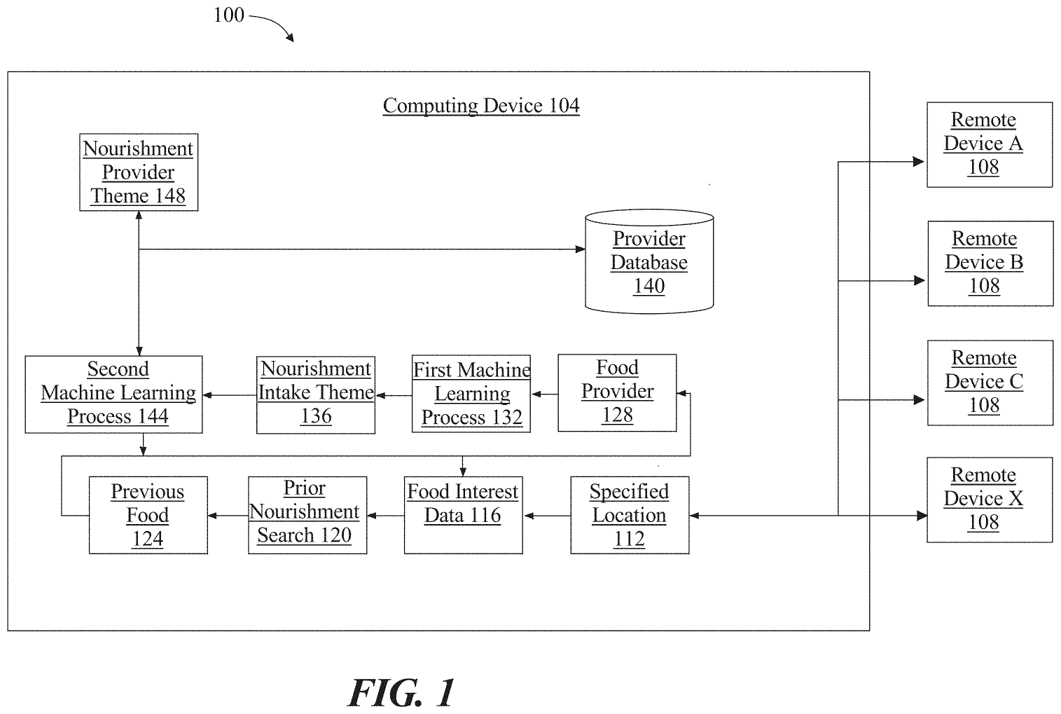

Referring now to FIG. 1, an exemplary embodiment of a system 100 for connecting food interests with food provider is illustrated. System 100 includes a computing device 104. Computing device 104 may include any computing device 104 as described in this disclosure, including without limitation a microcontroller, microprocessor, digital signal processor (DSP) and/or system on a chip (SoC) as described in this disclosure. Computing device 104 may include, be included in, and/or connect with a mobile device such as a mobile telephone or smartphone. Computing device 104 may include a single computing device 104 operating independently or may include two or more computing device 104 operating in concert, in parallel, sequentially or the like; two or more computing devices 104 may be included together in a single computing device 104 or in two or more computing devices 104. Computing device 104 may interface or connect with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an association, a building, a campus or other relatively small geographic space), a telephone network, a data network associated with a telephone/voice provider (e.g., a mobile communications provider data and/or voice network), a direct connection between two computing devices 104, and any combinations thereof. A network may employ a wired and/or a wireless mode of communication. In general, any network topology may be used. Information (e.g., data, software etc.) may be transmitted to and/or from a computer and/or a computing device 104. Computing device 104 may include but is not limited to, for example, a computing device 104 or cluster of computing devices 104 in a first position and a second computing device 104 or cluster of computing devices 104 in a second position. Computing device 104 may include one or more computing devices 104 dedicated to data storage, security, dispersal of traffic for load balancing, and the like. Computing device 104 may distribute one or more computing tasks as described below across a plurality of computing devices 104 of computing device 104, which may operate in parallel, in series, redundantly, or in any other manner used for dispersal of tasks or memory between computing devices 104. Computing device 104 may be implemented using a “shared nothing” architecture in which data is cached at the operative, in an embodiment, this may enable scalability of system 100 and/or computing device 104.

Continuing to refer to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence recurrently until a desired or commanded outcome is achieved; repetition of a step or a sequence of steps may be performed iteratively and/or recursively using outputs of previous repetitions as inputs to subsequent repetitions, assembling inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, computing device 104 is configured to receive, from a first remote device of a plurality of remote devices located in a specified location, an input including food interest data. A “remote device,” as used in this disclosure, is any additional computing device, such as a mobile device, laptop, desktop, computer, and the like. A “specified location,” as used in this disclosure, is a particular geographical place and/or position. A specified location 112 may be based on a global positioning system (GPS) of a user and/or a remote device 108 operated by the user. A specified location 112 may include the latitude and/or longitude of a position where a user and/or a remote device 108 operated by the user is currently location, and/or a position where the user and/or the remote device 108 operated by the user may be located in the future. Computing device 104 is configured to receive information from each of a plurality of remote device 108 in a specified location 112 utilizing any network methodology as described herein. “Food interest data,” as used in this disclosure, is a description of any eating habits and/or eating preferences that a user has. Eating habits may include any information pertaining to how many meals a user consumes each day, types of meals that a user consumes each day, amount of food consumed by a user at each meal, times of the day when a user eats meals each day, eating patterns, ingredient quality of meals, cooking habits of a user, food preparation habits of a user, fiscal limits and/or fiscal habits on meals, cuisine styles that a user prefers, and the like. Eating preferences may include any meals that a user likes or dislikes, any foods that a user likes or dislikes, any food allergies that a user has, any food intolerances that a user has, any foods that a user avoids, any diets and/or specialized way of eating that a user engages in, and the like. For instance and without limitation, food interest data 116 may include a description that a user follows a low-purine diet because of a previously diagnosed condition of gout, and as such the user avoids red meat, lamb, pork, organ meats, seafood, grain liquor, and high fructose corn syrup. In yet another non-limiting example, food interest data 116 may contain a description that a user follows a vegan diet and does not consume any animal ingredients because of ethical beliefs against consuming animals. In yet another non-limiting example, food interest data 116 may contain an indication that a user does not consume onions because the user does not like the taste of onions.

With continued reference to FIG. 1, food interest data 116 includes a prior nourishment search datum 120. A “prior nourishment search datum,” as used in this disclosure, is data including any numerical, character, and/or symbolic data containing information describing a user's web browsing history. Web browsing history includes any web pages that a user has visited including any associated data containing page titles visited, time of visit, day of visit, and the like. For instance and without limitation, a prior nourishment search datum 120 may contain information pertaining to a user's search for “foods to eat with irritable bowel syndrome.” In yet another non-limiting example, a prior nourishment search datum 120 may contain information describing a website that a user visited of a cafe where the user visited the menu page on the website for twenty minutes. In yet another non-limiting example, a prior nourishment search datum 120 may contain information describing a search a user performed looking for “Japanese establishments located within ten miles of my location.” A prior nourishment search datum 120 may be shared with computing device 104 utilizing any network methodology as described herein.

With continued reference to FIG. 1, food interest data 116 includes a previous food provider acquisition 124. A “previous food provider acquisition,” as used in this disclosure, is information describing any previous dining experiences a user had with a food provider 128. A “food provider,” as used in this disclosure, is any provider of food and/or beverages intended for human consumption. A food provider 128 may include a café, a grocery store, a food hall, a food truck, a test kitchen, a meal-maker, a meal-delivery kit, a homemade meal prepared by a family member, friend, co-worker, acquaintance, and the like. A food provider 128 may provide for sale and/or purchase any food and/or beverages. A food provider 128 may contain sit down dining options, take-out dining options, meal delivery, online ordering, and the like. A previous food provider acquisition 124 includes any previous dining experiences a user had with a food provider 128, such as any meals a user ordered from a food provider 128, the number of times that a user visited a food provider 128 and/or consumed meals from a food provider 128 over a certain time frame and the like. For instance and without limitation, a previous food provider acquisition 124 may describe that a user visited a Greek cafe for lunch six times in the past month. In yet another non-limiting example, a previous food provider acquisition 124 may contain a description of a meal that a user ordered at an establishment when dining out with friends.

With continued reference to FIG. 1, food interest data 116 includes a diagnosis. A “diagnosis” as used in this disclosure, is the identification of the nature of an illness or other problem by examination of various signs, and/or symptoms. A diagnosis may contain a user reported diagnosis provided by a professional. A “professional,” as used in this disclosure, is any person licensed or certified to provide health care services to natural persons. A professional may include but is not limited to a physician, a dentist, a nurse, a chiropractor, an optometrist, a physical or occupational therapist, a social worker, a clinical dietician, a clinical psychologist, a licensed professional counselor, a licensed marriage and family therapist, a pharmacist, a speech therapist, and the like. For instance and without limitation, food interest data 116 may contain a diagnosis such as rheumatoid arthritis, that a user was diagnosed with five year previously. Food interest data 116 may contain a description of one or more dietary patterns and/or ways of eating that a user has adopted due to a diagnosis. For instance and without limitation, food interest data 116 may contain a description that a user has coronary artery disease, and as such, the user is following a vegan diet. In yet another non-limiting example, food interest data 116 may contain a description that a user was previously diagnosed with multiple sclerosis, and the user follows a ketogenic diet to help manage multiple sclerosis. A diagnosis may contain a self-diagnosis, which may include a diagnosis made by a user. For example, a self-diagnosis may include a user who may self-diagnose a food intolerance after following an elimination diet and noticing symptoms that include bloating, gas, and diarrhea after consuming tomatoes. In yet another non-limiting example, a self-diagnosis may include a minor condition that may not be serious and that may be easily treatable with over the counter medications, such as a condition as head lice, skin abrasions, menstrual cramps, headache, or the common cold.

With continued reference to FIG. 1, computing device 104 receives food interest data 116 utilizing a questionnaire. A “questionnaire,” as used in this disclosure, is an instrument containing a series of questions and/or other prompts for information regarding a user's eating habits. A questionnaire may contain a series of one or more open ended questions that may allow a user to type or write in an answer to a prompt for information. For example, a questionnaire may ask how many times a user ate out at an eatery in the past thirty days, what eateries the user ate out at, and what the user ordered at the eateries that the user ate out at. A questionnaire may contain a series of questions and/or prompts for information that may contain multiple answers that a user can choose to select such as to circle all foods that a user consumes on a daily basis, or to select one or more foods and/or beverages that a user does not like and doesn't consume frequently. A questionnaire may contain a series of photographs of various foods and/or meals and ask a user to selection photographs of foods and/or meals that a user enjoys eating, and to selection photographs of foods and/or meals that a user does not enjoy eating.

With continued reference to FIG. 1, computing device 104 is configured to generate as a function of a first machine-learning process 132, a nourishment intake theme for the first remote device, wherein the first machine-learning process 132 utilizes food interest data as an input, and outputs the nourishment intake theme. A “machine-learning process,” as used in this disclosure, is a process that automatedly uses a body of data known as “training data” and/or a “training set” to generate an algorithm that will be performed by computing device 104 and/or a module to produce outputs given data provided as inputs; this is in contrast to a non-machine learning software program where the commands to be executed are determined in advance by a user and written in a programming language. “Training data,” as used in this disclosure, is a set of examples that contain pairs of an input and a corresponding output, which are used to model relationships between two or more categories of data elements. Training data may be formatted to include labels, for instance by associating data elements with one or more descriptors corresponding to categories of data elements. Training data may not contain labels, where training data may not be formatted to include labels. A machine-learning process may include calculating one or more machine-learning algorithms and/or producing one or more machine-learning models. A machine-learning process may include a supervised machine-learning process that applies learned associations from the past to new data using labeled training data to predict future events. A supervised machine-learning process produces an inferred function to make predictions about output values. A supervised machine-learning process may include for example, active learning, classification, regression, and/or similarity learning. A machine-learning process may include an unsupervised machine-learning process where training data utilized to train the unsupervised machine-learning process may not be classified or labeled. An unsupervised machine-learning process may infer a function to describe a hidden structure from unlabeled data. An unsupervised machine-learning process may include for example, clustering, anomaly detection, neural networks, latent variable models, and the like. A machine-learning process may include a semi-supervised machine-learning process that may utilize a combination of both labeled and unlabeled training data. A semi-supervised machine-learning process may include generative models, low density separation, graph-based methods, heuristic approaches, and the like. A “nourishment intake theme,” as used in this disclosure, is the identification of foods and/or beverages that a user habitually eats. A nourishment intake theme 136 may identify a particular diet that a user follows, such as a user who follows a gluten free diet, or a user who follows a dietary approach to stop hypertension (DASH) diet. A nourishment intake theme 136 may identify any foods and/or beverages that a user is not allowed to consume, such as a user with diabetes who is not allowed to consume any high fructose corn syrup. A nourishment intake theme 136 may identify recommended serving sizes of foods and/or nutrients that a user should consume, such as a user with pre-diabetes who is recommended to consume no more than 50 grams of carbohydrates each day. A nourishment intake theme 136 may identify one or more nutrients and/or minerals that a user may require, such as a user with hypothyroidism who may require additional iodine.

With continued reference to FIG. 1, computing device 104 generates a first machine-learning process 132 that utilizes food interest data 116 as an input, and outputs a nourishment intake theme 136 for each of a plurality of remote device 108. Computing device 104 trains a first machine-learning process 132 utilizing training data, including any of the training data as described herein. Training data may be obtained from previous iterations of generating a first machine-learning process 132, user inputs and/or questionnaire responses, expert inputs, and the like. Computing device 104 generates a first machine-learning process 132 utilizing a clustering algorithm. A “clustering algorithm,” as used in this disclosure, is a machine-learning process that groups a set of objects that are more similar to each other to produce a cluster, than to those in other groups or clusters. A clustering algorithm may include generating one or more clustering models that may include but are not limited to connectivity models such as hierarchical clustering that builds models based on distance connectivity, centroid models such as k-means clustering that represent each cluster by a single mean vector, distribution models that contain clusters modeled using statistical distributions such as multivariate normal distributions, density models such as density based spatial clustering (DBSC) and ordering points to identify the clustering structure (OPTICS) which generate clusters as connected dense regions in data space, sub-space models such as biclustering where clusters are modeled with both cluster members and relevant attributes, group models, graph based models, signed graph models, neural models, and the like. A clustering algorithm generates a set of clusters, that contain all objects in a data set. A clustering algorithm may specify the relationship of clusters to each other. A clustering algorithm may generate hard clusters, where each object belongs to a cluster or not. A clustering algorithm may generate soft clusters, where each object belongs to each cluster to a certain degree. A clustering algorithm may include a strict partitioning cluster, where each object belongs to exactly one cluster. A clustering algorithm may include a strict partitioning cluster with outliers, where objects may not belong to any cluster, and may be considered an outlier. A clustering algorithm may include overlapping clustering, where objects may belong to more than one cluster. A clustering algorithm may include hierarchical clustering, where objects that belong to a child cluster also belong to a parent cluster. A clustering algorithm may include subspace clustering, where clusters do not overlap.

With continued reference to FIG. 1, computing device 104 is configured to detect a plurality of food provider 128 located within a specified location 112, wherein each of the plurality of food providers provides a nourishment provision. A food provider 128 includes any of the food provider 128 as described above in more detail. A food provider 128 may include for example, an eatery such as a Thai eatery located within a specified location 112. A food provider 128 may include a meal maker such as a home cook that prepares and sells meals to users within a specified location 112. A food provider 128 may be located within a specified location 112, when the food provider 128 is physically located within a specified location 112 and/or when a food provider 128 delivers and/or provides service within a specified location 112. Computing device 104 detects a plurality of food provider 128 by locating food provider 128 within a certain longitude and latitude distance of a specified location 112. Computing device 104 contains a provider database 140 that contains information pertaining to food provider 128 and the location of food provider 128 and areas that they service. Provider database 140 may be implemented, without limitation, as a relational database, a key-value retrieval datastore such as a NO SQL database, or any other format or structure for use as a datastore that a person skilled in the art would recognize as suitable upon review of the entirety of this disclosure. Such information may be updated utilizing any network methodology as described herein. For instance and without limitation, provider database 140 may contain an entry containing a description of a juice bar that has a physical location in Vero Beach, Fla., and which delivers to locations that include Fort Pierce, Port Saint Lucie, Stuart Sebastian, and Cocoa Beach. Computing device 104 detects a plurality of food provider 128 such as by generating a query to locate food provider 128 listed within provider database 140, to identify those who are located within and/or provide service to a specified location 112. A “query,” as used in this disclosure, is any information utilized to detect a food provider 128. A query may be generated utilizing information about a specified location 112. For instance and without limitation, computing device 104 may generate a query to detect a food provider 128 located within the greater Miami area, when a specified location 112 identifies the East Coast of Florida. In yet another non-limiting example, computing device 104 may generate a query to detect a food provider 128 located in Anchorage, Ala. when a specified location 112 identifies southern Alaska. Food provider 128 stored within provider database 140 may be organized by locations in which they are located and/or serve.

With continued reference to FIG. 1, computing device 104 detects a plurality of food provider 128 using nourishment provisions available at food provider 128. A “nourishment provision,” as used in this disclosure, is any food items available for purchase and/or sale offered by a food provider 128. A nourishment provision, may include a menu item, such as a list of meals and/or beverages available to purchase from a nourishment provision. For example, a menu items may contain meal options available for breakfast, lunch, dinner, and/or snacks, such as breakfast menu items that contains choices including a Mediterranean omelet, a berry smoothie, gluten free pancakes, a vegan yogurt parfait, and steel-cut gluten free oatmeal. A nourishment provision may include a list of one or more meal items available for purchase only at certain times of the day and/or only on specified days such as various special meals that may be available when certain foods are in season and/or available in various locations. For instance and without limitation, a nourishment provision may include a dinner entrée that contains King Salmon may only be available at a nourishment location from mid-May to early June, when King Salmon is in season. Computing device 104 locates a plurality of food provider 128 by evaluating nourishment provisions available by food provider 128. For instance and without limitation, a nourishment intake theme 136 that labels a user as following a vegetarian nourishment intake theme 136 may be utilized to eliminate nourishment provisions that contain seafood and/or meat. In yet another non-limiting example, a nourishment intake theme 136 label that specifies a user as following a Mediterranean style of eating may be utilized to locate nourishment provisions that contain Mediterranean foods including vegetables, fruits, whole grains, fish, nuts, seeds, and olive oil. Computing device 104 detects food provider 128 by searching food provider 128 contained within provider database 140. For instance and without limitation, computing device 104 generates a query to first locate food provider 128 located throughout the state of Rhode Island, and to detect those food providers 128 located throughout Rhode Island that provide nourishment provisions that contain dairy free nourishment provisions. Computing device 104 detects a plurality of food provider 128 by examining previous food provider acquisition 124, to identify previous food provider 128 a user interacted with and/or acquired meals from.

With continued reference to FIG. 1, computing device 104 generates as a function of a second machine-learning process 148, a nourishment provider theme 144 for each of a plurality of food provider 128 located within a specified location 112. A “nourishment provider theme,” as used in this disclosure, is a label identifying cuisines and/or diets that a food provider 128 can create nourishment provisions for. A nourishment provider theme 144 may identify a certain cuisine that nourishment provisions offered by a food provider 128 fall into, such as Japanese, American comfort food, Korean cuisine, Mexican cuisine, and the like. In yet another non-limiting example, a nourishment provider theme 144 may identify one or more nourishment intake theme 136 that a food provider 128 can prepare meals for, such as by modifying and/or substituting currently available nourishment provisions. For instance and without limitation, a Japanese eatery that offers nourishment provisions that contain sushi rolls made with rice, may be able to modify the nourishment provisions to provide grain free sushi rolls made with cauliflower rice instead of traditional rice. In yet another non-limiting example, a seafood eatery that offers entrees that contain freshly caught seafood may be able to modify the nourishment provisions to offer vegetarian and/or vegan entrees that do not contain any freshly caught seafood. Information pertaining to the availability of ingredients to be substituted and/or nourishment provisions to be modified to comply with particular nourishment intake theme 136 may be stored within provider database 140.

With continued reference to FIG. 1, a second machine-learning process 148 includes any machine-learning process suitable for use as a first machine-learning process 132 as described above in more detail in reference to FIG. 1. A second machine-learning process 148 utilizes a nourishment provision as an input, and outputs a nourishment provider theme 144. Second machine-learning process 148 is calculated utilizing a classification algorithm. A “classification algorithm,” as used in this disclosure, is a machine-learning model that sorts inputs into categories or bins of data. A classification algorithm may include linear classifiers such as Fisher's linear discriminant, logistic regression, Naïve Bayes classifier, perceptron, support vector machine, quadratic classifier, kernel estimation, k-nearest neighbor, boosting, random forest decision tree, neural network, and/or learning vector quantization. A classification algorithm is trained by computing device 104 utilizing any training data as described herein. Training data is obtained from previous iterations of calculating classification algorithm, expert inputs, user inputs and the like. Classification algorithm utilizes a nourishment provision as an input and outputs a nourishment provider theme 144.

With continued reference to FIG. 1, computing device 104 is configured to compare a remote device 108 having a nourishment intake theme 136 with a nourishment intake theme 136. Comparing includes determining if a nourishment provider theme 128 meets dietary recommendations contained within a nourishment intake theme 136. For instance and without limitation, a food provider 128 that offers ketogenic entrees may compare a nourishment intake theme 136 containing a ketogenic diet. In yet another non-limiting example, a food provider 128 that offers paleo entrees may compare a nourishment intake theme 136 including paleo nourishment themes, low-carbohydrate nourishment intake theme 136, grain free nourishment intake theme 136, and dairy free nourishment intake theme 136. Connecting includes identifying a food provider 128 that offers nourishment provisions that compare a nourishment intake theme 136 related to a remote device 108 with a remote device 108. Comparing includes identifying a food provider 128 that offers nourishment provisions that fit one or more nourishment intake theme 136. Comparing may include generating a list of food provider 128 that compare to one or more remote device 108. For instance and without limitation, computing device 104 may identify a food provider 128 that contains a nourishment provider theme 144 of gluten free meals that compares nourishment intake theme 136 that include gluten free, wheat free, standard American diet, rye free, vegetarian, pescatarian, and the like.

With continued reference to FIG. 1, computing device 104 supports transmission of a nourishment provision as a function of comparing a first nourishment intake theme to a nourishment provider theme. Supporting transmission includes using any network methodology as described herein to share with a remote device 108 information that identifies a food provider 128 that compares a nourishment intake theme 136 identified for the remote device 108. Supporting transmission includes sharing advertising material with a remote device 108 utilizing any network methodology as described herein. Advertising material includes any data transmitted identifying a food provider 128. For instance and without limitation, computing device 104 supports transmission of data to a remote device 108, identifying three food provider 128 that compares a nourishment identified for the remote device 108. Computing device 104 supports transmission of data to a remote device 108 utilizing any network methodology as described herein. Computing device 104 is configured to support transmission of default food provider located within a specified location 112. A “default food provider,” as used in this disclosure, is any food provider 128 that is located within a specified location 112, but that does not contain a nourishment provision that compares a nourishment intake theme 136 identified for a remote device 108. For instance and without limitation, a remote device 108 that contains a nourishment intake theme 136 such as a vegetarian theme may receive default food provider located within a specified location 112 of the remote device 108, but the food provider 128 may not necessarily offer nourishment provisions that comply with the vegetarian theme. Computing device 104 is configured to block transmission of nourishment provisions that fall outside a nourishment intake theme 136. Nourishment provisions fall outside a nourishment intake theme 136 when the nourishment provisions do not compare the nourishment intake theme 136. For instance and without limitation, a food provider 128 may offer five dinner entrees, of which three entrees compare with a nourishment intake theme 136 and two entrees do not compare with the nourishment intake theme 136. In such an instance, computing device 104 supports transmission of the three entrees that compare the nourishment intake theme 136, and computing device 104 blocks transmission of the two entrees that do not compare the nourishment intake theme 136. Advertising material is discussed in further detail below.

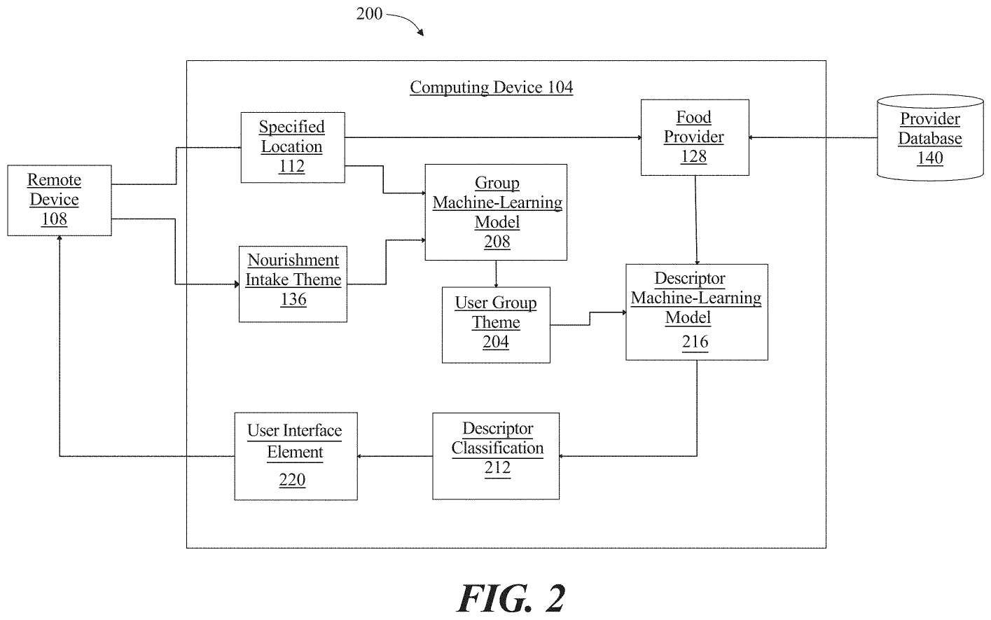

Referring now to FIG. 2, an exemplary embodiment of a system 200 for generating textual outputs based on descriptor classifications is presented. In some embodiments, system 200 includes computing device 104. In embodiments, computing device 104 may be configured to receive an input from first remote device 108 of a plurality of remote devices. In embodiments, input from first remote device 108 may include specified location 112. In an embodiment, input from first remote device 108 may include nourishment intake theme 136.

Continuing to refer to FIG. 2, computing device 104, in an embodiment, may be configured to generate a user group theme 204 for first remote device 108. A “user group theme,” as used herein is a plurality of attributes related to one or more user groups that may apply to one or more remote devices 108. In some embodiments, user group theme 204 may be attributes that are common among two or more remote devices 108. In a nonlimiting example, user group theme 204 may be vegetarians whose remote devices are currently located in the Dumbo region of Brooklyn N.Y. In some embodiments, user group theme 204 may include action items. An “action item,” as used herein, is an event or activity that applies to a group of users. In a nonlimiting example, user group theme 204 may be a group of users currently located in the Dumbo area of Brooklyn N.Y. that are looking for a Cinco de Mayo celebration, which may assign Mexican food as the type of cuisine for that group. In an embodiment, computing device 104 may be further configured to generate user group theme 204 as a function of a group machine-learning model 208. In some embodiments, group machine-learning model 208 may receive nourishment intake theme 136 and specified location 112 as input, and may output user group theme 204. Group machine learning model 208 may be a classifier. Classifier may include any classifier described herein. Group machine learning model 208 may include any machine learning methodologies described throughout this disclosure. Group machine learning model 108 may be trained using training data. Training data may include past iterations of group machine learning model 208. In some embodiments, training data may include correlations of nourishment intake themes and locations to user groups. In some embodiments, training data may include inputs by users. In some embodiments, training data may include mock data. As used herein, “mock data” is data generated for the purpose of training a machine learning model. Training data may include any training data described herein. Training data may include prior iterations of group machine-learning model.

Still referring to refer to FIG. 2, in some embodiments, computing device 104 may be further configured to identify at least a food provider 128 located within specified location 112. In embodiments, at least a food provider 128 may be included in provider database 140. In some embodiments, computing device 104 may be further configured to identify at least a food provider 128 as function of a bid ranking. A “bid ranking,” as used herein, is the monetary amount a food provider 128 is willing to pay operator of system 200. In a nonlimiting example, when two or more food providers 128 are identified for the specified location 112, the food provider 128 willing to pay the most amount of money is selected to be presented to a user. Identifying at least a food provider may be consistent with disclosure in reference to FIG. 1.

With continued reference to FIG. 2, in some embodiments, computing device 104 may be configured to determine a descriptor classification 212 for first remote device 108. A “descriptor classification,” as used herein, is a data structure that organizes two or more entities into categories based on predefined criteria. In some embodiments, descriptor classification 212 may be represented as list of categories, each representing a classification. In embodiments, descriptor classification 212 may include data fields or attributes associated with each category or classification. In embodiments, descriptor classification 212 may be included in a database. In an embodiment, descriptor classification 212 may include an advertisement theme. An “advertisement theme,” as used herein is a type of advertisement for at least a food provider 128 that applies to a user group theme 204. In a further nonlimiting example, descriptor classification 212 may be an advertisement for vegetarian restaurants located in the Dumbo area of Brooklyn N.Y.. In some embodiments, computing device 104 may be configured to generate descriptor classification 212 as a function of a descriptor machine-learning model 216. Descriptor machine-learning model 216 may include any machine learning model described throughout this disclosure. Descriptor machine learning model may include a classifier. Classifier may include any classifier described throughout this disclosure. Descriptor machine-learning model 216 may be trained with training data consistent with training data described in this disclosure. Training data may include prior iterations of advertisement machine-learning model 216. Training data may include mock data. In some embodiments, descriptor machine-learning model 216 may receive at least a food provider 128 and user group theme 204 as input and may output descriptor classification 212. In another nonlimiting example, descriptor machine learning model 216 may receive an input of “students at college X located in city A” as an input, and output a descriptor classification 212 that includes advertisements targeted at colleges students, such as pizza or other fast-food items, from food provider 128 located in city A.

Continuing to refer to FIG. 2, in some embodiments, computing device 104 may be configured to generate a user interface element 220 at first remote device 108 as a function of the descriptor classification 212. In embodiments, user interface element 220 may include one or more elements of a graphical user interface (GUI). In some embodiments, user interface element 220 may be configured to display a plurality of user interface elements such as texts, images, videos, buttons, forms, menus, progress indicators, alerts, tabs, windows, and the like. In a nonlimiting example, at least a user interface element 220 may include an advertisement for a food provider 128, where the user interface elements 220 may configure first remote device to display an image related to items of food provider 128 and a button which may configure first remote device 108 to generate an “http” connection for a website related to food provider 128. An “http connection,” as used herein, is a communication protocol used for transferring data over the internet. In some embodiments, user interface element 220 may configure first remote device 108 to interact with a user through touch-based inputs. Touch-based inputs may include swiping, pinching, and the like. In embodiments, user interface element 220 may include information identifying at least a food provider 128 and the type of food served. In some embodiments, user interface element 220 may include instructions configuring first remote device 108 to modify property of GUI components such as size, color, alignment, spacing, and the like. In some embodiments, user interface element b220 may include instructions modifying a behavior of a GUI in first remote device 108. Modifying GUI behavior may include making changes to application logic in first remote device 108 such as by adding or removing event listeners, updating data bindings, and the like. In a nonlimiting example, user interface element 220 may include the name and location of a food provider 128, and information related to their menu, such as gluten free options for some that has low tolerance for gluten. It will be apparent to one of ordinary skill in the art, upon reading this disclosure, that these descriptions are provided as way of example and that user interface element 220 may modify one or more remote devices in ways not described herein.

Still referring to FIG. 2, in embodiments, computing device 104 may be configured to transmit at least a user interface element 220 to remote device 108. In embodiments, computing device 104 may be configured to transmit at least a user interface element 220 to a second remote device. In embodiments, computing device 104 may be configured to transmit at least a user interface element 220 to a plurality of remote devices. In an embodiment, computing device 104 may be further configured to receive interface feedback. An “advertisement feedback,” as used herein, is feedback related to the interface element 220 transmitted. In a nonlimiting example, advertisement feedback may be a rating of how effective the advertisement was. In further embodiments, user interface element 220 may be transmitted to other remote devices 108 based on advertisement feedback. In a nonlimiting example, user interface element 220 may be sent to other remote devices within same user group theme 204. User interface element 220 may be stored in a database.

With continued reference to FIG. 2, computing device may be further configured to receive food interest data 116 from first remote device 108 and generate an interest group theme as a function of an interest machine-learning model. An “interest group theme,” as used herein, is a grouping of remote devices that includes a commonality of interest data 116. In some embodiments, interest machine-learning model may receive food interest data 116 and specified location 112 as inputs and may output interest group theme. In further embodiments, computing device 104 may be further configured to generate a targeted advertisement theme as a function of an interest machine-learning model. A “targeted advertisement theme,” as used herein are advertisements generated based on interest group theme. In further embodiments, targeted machine-learning model may receive at least a food provider 128 and the interest group theme as inputs and may output the targeted advertisement theme. In further embodiments, computing device 104 may be further configured to determine at least a user interface element 220 as function of the targeted advertisement theme.

Continuing to refer to FIG. 2, in some embodiments, computing device 104 may be configured to receive an availability datum. An “availability datum,” as used herein, is an element of data identifies the availability of a service, or the type of food being served. In another embodiment, availability datum may be how many orders a food provider 128 may be able to handle. In a nonlimiting example, a food provider 128 may put a pause on advertisements due to their kitchen being at capacity. In an embodiment, availability datum may be seasonal availability. Seasonal availability may be availability of locally grown food. In a nonlimiting example, availability datum for locally source corn in the Northeast of the United States may be between June and August. In embodiments, computing device 104 may be further configured to generate at least a user interface element 220 at first remote device 108 as a function of descriptor classification 212 and the availability datum.

Still referring to FIG. 2, in some embodiments, computing device 104 may be configured to receive a promotional datum. A “promotional datum,” as used herein, is information set by food providers 128 related to advertising. In a nonlimiting example, a food provider 128 may set specific time which advertisements are sent, such as a food provider 128 promoting happy hour deals. In some embodiments, promotional datum may include especial deals for new users. In some embodiments, computing device 104 may be further configured to determine at least a user interface element 220 for first remote device 108 as a function of descriptor classification 212 and the promotional datum.

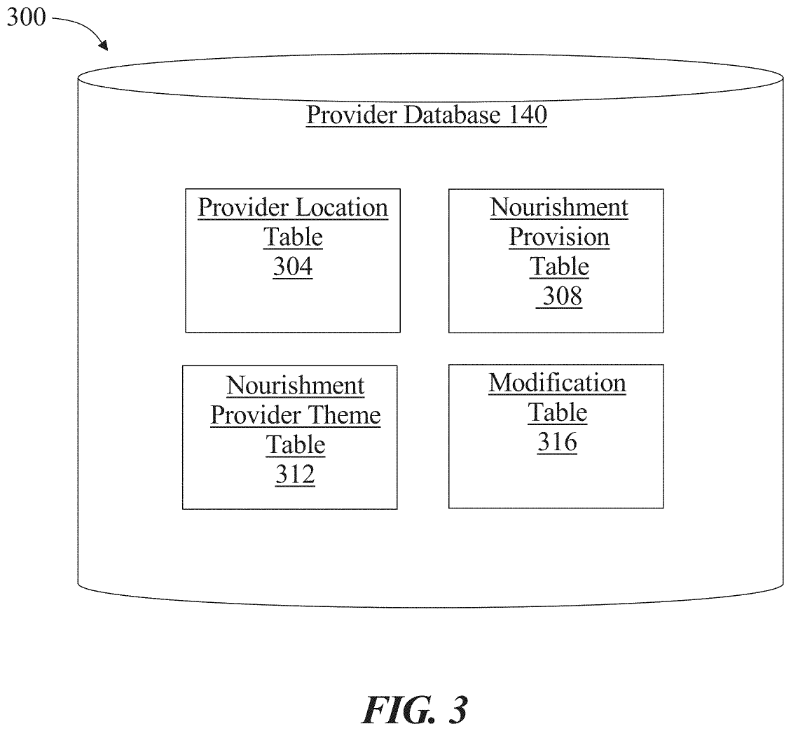

Referring now to FIG. 3, an exemplary embodiment 300 of provider database 140 is illustrated. Provider database 140 may be implemented as any data structure suitable for use as described above in more detail in reference to FIG. 1. One or more tables contained within provider database 140 may include provider location table 304; provider location table may contain information describing a specified location 112, containing information detailing where a food provider 128 is located and/or areas where a food provider 128 delivers to and/or provides services to. For instance and without limitation, provider location table 304 may contain information detailing that a food provider 128 is located in Oklahoma City, and provides service and/or delivery to surrounding towns that include Bethany, Yukon, Moore, McLoud, and Edmond. One or more tables contained within provider database 140 may include nourishment provision table 308; nourishment provision table 308 may contain information pertaining to nourishment provisions available at a food provider 128, including for example, one or more menu items. For instance and without limitation, nourishment provision table 308 may contain a list of nourishment provisions available at food provider 128 for lunch, which include a cobb salad, a Caesar salad, a turkey club sandwich, a California chicken sandwich, and a taco salad. One or more tables contained within provider database 140 may include nourishment provider theme table 312; nourishment provider theme table 312 may contain information describing a nourishment provider theme 144 of one or more food provider 128. For instance and without limitation, nourishment provider theme 144 table 312 may contain an entry describing a food provider 128 as having a nourishment provider theme 144 of gluten free and vegan Mediterranean style nourishment provisions. One or more tables contained within provider database 140 may include modification table 316; modification table 316 may contain information describing one or more modifications a food provider 128 can make regarding nourishment provisions and/or creating nourishment provisions that fit one or more nourishment provider theme 144. For instance and without limitation, modification table 316 may contain an entry describing a food provider 128 that has a nourishment provision that contains cod with risotto and vegetables that can be modified to create both a vegetarian and/or a vegan nourishment provision.

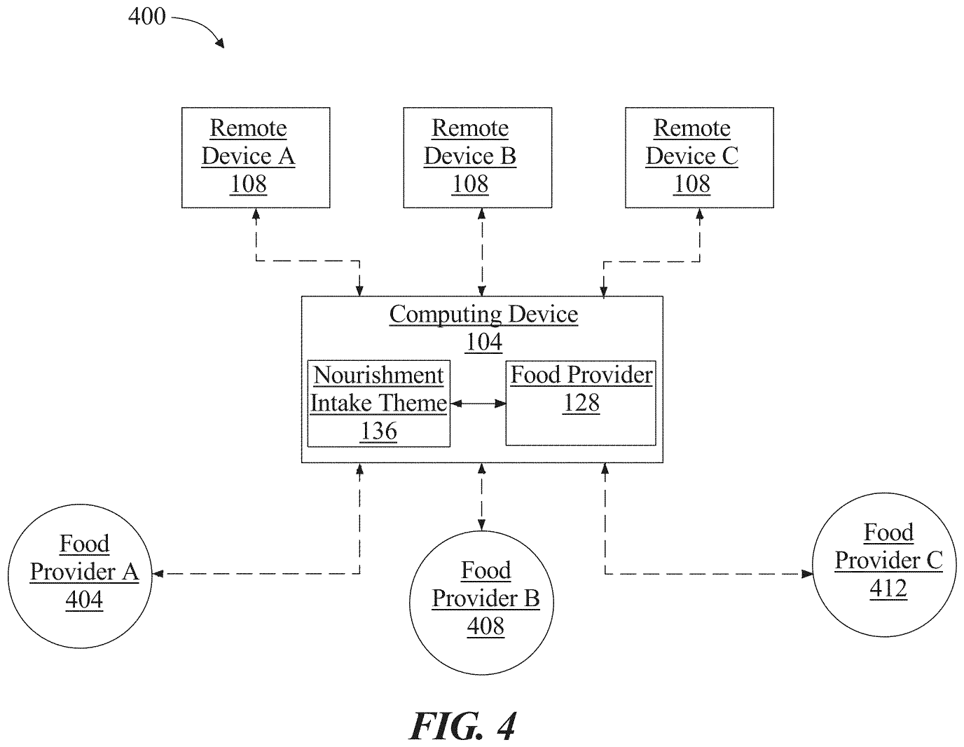

Referring now to FIG. 4, an exemplary embodiment of comparing nourishment intake theme 136 with food provider 128 is illustrated. Computing device 104 receives from each of a plurality of remote device 108 located in a specified location 112, a plurality of inputs containing food interest data 116, as described above in more detail in reference to FIG. 1. In an embodiment, a plurality of remote device 108, such as remote device 108 A, remote device 108 B, and remote device 108 C may be in communication with computing device 104. Computing device 104 generates as a function of a first machine-learning process 132, a nourishment intake theme 136 for each of a plurality of remote device 108. A first machine-learning process 132 includes any of the machine-learning processes as described above in more detail in reference to FIG. 1. Computing device 104 detects a plurality of food provider 128 located within a specified location 112 providing a nourishment provision. A nourishment provision includes any of the nourishment provisions as described above in more detail in reference to FIG. 1. For instance and without limitation, computing device 104 detects food provider 128 A 404, food provider 128 B 408, and food provider 128 C, 412, contained within a specified location 112. A specified location 112 includes any of the specified location 112 as described above in more detail in reference to FIG. 1, including a particular geographical region or location. Computing device 104 generates as a function of a second machine-learning process 148, a nourishment provider theme 144 148 for each of a plurality of food provider 128 located within a specified location 112. For instance and without limitation, computing device 104 identifies a first nourishment provider theme 144 for food provider A 128, a second nourishment provider theme 144 for food provider B 128, and a third nourishment provider theme 144 for food provider C 128. Second machine-learning process 148 includes any of the second machine-learning process 148 as described above in more detail in reference to FIG. 1. Second machine-learning process 148 utilizes a nourishment provision as an input, and outputs a nourishment provider theme 144. Computing device 104 compares a remote device 108 having a nourishment intake theme 136 with a food provider that offers a nourishment provision 416 that compares a nourishment intake theme 136. For instance and without limitation, computing device 104 may compare a nourishment provision 416 offered by food provider A 404 to a nourishment intake theme 136 of remote device 108 B. In such an instance, computing device 104 supports transmission of the nourishment provision compare 416 offered by food provider A 404 to remote device 108 B, utilizing any network methodology as described herein.

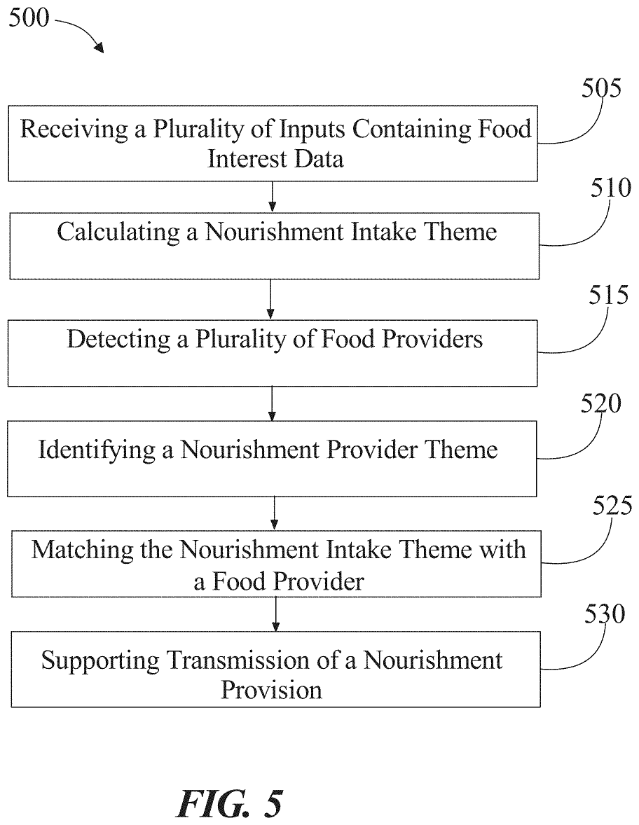

Referring now to FIG. 5, an exemplary embodiment of a method 500 for connecting food interests with food provider 128 is illustrated. At step 505, computing device 104 receives from a first remote device of a plurality of remote device 108 located in a specified location 112, a plurality of inputs containing food interest data 116. A remote device 108 includes any of the remote device 108 as described above in more detail in reference to FIG. 1. For instance and without limitation, a remote device 108 may include a mobile computing device. In yet another non-limiting example, a remote device 108 may include an additional computing device, including any device suitable for use as computing device 104 as described above in more detail in reference to FIG. 1. A specified location 112 includes any of the specified location 112 as described above in more detail in reference to FIG. 1. A specified location 112 includes a particular geographical place and/or position. For instance and without limitation, a specified location 112 may include a geographical place such as Boston, Mass. In yet another non-limiting example, a specified location 112 may include a GPS location, such as Coral Way Village, located in Westchester, Fla. Computing device 104 receives from each of a plurality of remote device 108, utilizing any network methodology as described herein, a plurality of inputs containing food interest data 116. Food interest data 116 includes any of the food interest data 116 as described above in more detail in reference to FIG. 1. Food interest data 116 contains a description of any eating habits and/or eating preferences that a user has. For instance and without limitation, food interest data 116 may contain an input specifying that a user consumes a raw foods diet, and consumes foods such as fresh fruits, raw vegetables, sprouted grains, nut and seed butter, and cold pressed coconut oil. In yet another non-limiting example, food interest data 116 may contain an input specifying that a user only eats meals between the hours of 11:00 am and 4:00 pm every day, the other hours of the day the user engages in intermittent fasting.

With continued reference to FIG. 5, food interest data 116 includes a prior nourishment search datum 120. A prior nourishment search datum 120 includes any of the prior nourishment search datum 120 as described above in more detail in reference to FIG. 1. A prior nourishment search datum 120 may include a user's web browsing history, including for example any food provider 128 that a user may have search for, or any questions relating to food provider 128 that a user researched. For instance and without limitation, a prior nourishment search datum 120 may contain a web browsing search that a user entered looking for “recommended barbeque eateries located in Dallas, Tex.” In yet another non-limiting example, a prior nourishment search datum 120 may contain a web browsing history that contains an input describing that a user visited a website for a vegetarian eatery and browed through the online menu for twenty seven minutes. Food interest data 116 contains a previous food provider acquisition 124. A previous food provider acquisition 124 includes any of the previous food provider acquisition 124 as described above in more detail in reference to FIG. 1. A previous food provider acquisition 124 may contain a list of all meals that a user consumed over the course of the previous two weeks. In yet another non-limiting example, a previous food provider acquisition 124 may contain a description of a meal that a user consumed from a food provider 128 during the previous week. Food interest data 116 includes a diagnosis. A diagnosis includes any of the diagnoses as described above in more detail in reference to FIG. 1. A diagnosis may include a description of a medical condition that a user was diagnosed with by a medical professional. For instance and without limitation, a diagnosis may contain a description of a medical condition such as ulcerative colitis that a user was diagnosed with three years prior, by a medical professional such as a gastroenterologist. A diagnosis may include information pertaining to a self-diagnosis made by a user, such as when a user may self-diagnose a self-limiting condition that resolves on its own without treatment or can be treated with one or more over the counter treatment options. For instance and without limitation, a diagnosis may include a self-limiting condition such as a migraine headache, that a user treated with a caffeine pill.

With continued reference to FIG. 5, computing device 104 receives information pertaining to food interest data 116 utilizing a questionnaire. A questionnaire includes any of the questionnaires as described above in more detail in reference to FIG. 1. For instance and without limitation, computing device 104 may transmit to a remote device 108 a questionnaire that includes a list of hundreds of the most commonly diagnosed medical conditions and ask a user to select any of the diagnosed medical conditions that the user has previously and/or is currently diagnosed with. In yet another non-limiting example, a questionnaire may include a prompt for information from a user, whereby a questionnaire may display photographs of individual foods and/or meals, and ask a user to select any foods and/or meals that a user likes to consume, and to select any foods and/or meals that a user dislikes to consume.

With continued reference to FIG. 5, at step 510, computing device 104 generates as a function of a first machine-learning process 132, a nourishment intake theme 136 for each of the plurality of remote device 108. A nourishment intake theme 136 identifies foods and/or beverages that a user habitually eats, as described above in more detail in reference to FIG. 1. For instance and without limitation, a nourishment intake theme 136 may identify a particular eating pattern that a user follows based on foods and/or beverages that a user habitually consumes. For example, a user who consumes foods that include meats, vegetables, fruits, and dairy free milk may be identified as having a nourishment intake theme 136 of paleo. In yet another non-limiting example, a user who consumes high-fats, moderate proteins and very low carbohydrates may be identified as having a nourishment intake theme 136 of ketogenic. A nourishment intake theme 136 is identified by calculating a first machine-learning process 132. A first machine-learning process 132 includes any of the machine-learning processes as described above in more detail in reference to FIG. 1. A first machine-learning process 132 includes calculating a clustering algorithm. A clustering algorithm may be implemented as any of the clustering algorithms as described above in more detail in reference to FIG. 1.

With continued reference to FIG. 5, at step 515, computing device 104 detects a plurality of food provider 128 located within a specified location 112 providing a nourishment provision. Computing device 104 detects a plurality of food provider 128, by finding food provider 128 that are located within and/or provide service within a specified location 112. Computing device 104 generates a query to locate food provider 128 contained within provider database 140 that offer nourishment provisions within a specified location 112. For instance and without limitation, a specified location 112 may specify San Diego, Calif. In such an instance, computing device 104 generates a query to detect food provider 128 that are located within San Diego, and/or that deliver and/or provide service to users located within San Diego. Food provider 128 may be listed and contained within provider database 140 based on specified location 112 information as described above in more detail in reference to FIG. 1. Computing device 104 detects a plurality of food provider 128 located within a specified location 112 by locating nourishment provisions available at food provider 128. Information pertaining to nourishment provisions available at food provider 128 may be stored within provider database 140 and may be updated utilizing any network methodology as described herein. Computing device 104 may examine nourishment provisions to examine if any of the nourishment provisions comply with a nourishment provider theme 144, and/or can be modified to comply with the nourishment provider theme 144. For instance and without limitation, a food provider 128 that offers nourishment provisions that are all cooked in peanut oil may be unable to modify a nourishment provision that is not cooked in peanut oil and/or that is not cross-contaminated with peanut oil. In such an instance, computing device 104 may eliminate such a food provider 128. In yet another non-limiting example, a food provider 128 that offers nourishment provisions that include grain free sushi rolls may be detected as providing a nourishment provision that complies with a low-carbohydrate nourishment intake theme 136 for a user with pre-diabetes who is significantly limiting intake of carbohydrates.

With continued reference to FIG. 5, at step 520, computing device 104 generates as a function of a second machine-learning process 148 a nourishment provider theme 144 for each of a plurality of food provider 128 located within a specified location 112. A nourishment provider theme 144 includes any of the nourishment provider theme 144s as described above in more detail in reference to FIG. 1. A nourishment provider theme 144 identifies cuisines and/or diets that a food provider 128 can create nourishment provisions for. For instance and without limitation, a nourishment provider theme 144 may identify a food provider 128 that offers nourishment provisions that are grain free, vegan, dairy free, gluten free, and vegetarian. In yet another non-limiting example, a nourishment provider theme 144 may identify a food provider 128 that offers Mexican nourishment provisions that can be modified to be dairy free. Information pertaining to modifications of nourishment provider theme 144s may be stored within provider database 140 as described above in more detail in reference to FIG. 2. A food provider 128 may contain a nourishment provider theme 144 that identifies more than one cuisine, diets, and/or eating patterns that nourishment provisions may be modified for and/or apply to. For instance and without limitation, a food provider may prepare that are gluten free, nut free, soy free, vegetarian, and onion free. Second machine-learning process 148 includes any of the machine-learning processes as described above in more detail in reference to FIG. 1. Second machine-learning process 148 may include generating a classification algorithm, including any of the classification algorithms as described above in more detail in reference to FIG. 1. Second machine-learning process 148 utilizes a nourishment provision as an input, and outputs a nourishment provider theme 144.

With continued reference to FIG. 5, at step 525, computing device 104 compares a first remote device 108 having a first nourishment intake theme 136 with a nourishment provider theme 128. Comparing includes identifying food provider 128 located within a specified location 112 that offer nourishment provisions that meet and/or exceed a nourishment intake theme 136. Comparing includes examining a nourishment provider theme 144 to determine if a nourishment provider theme 144 can be adjusted to accommodate a nourishment intake theme 136. For instance and without limitation, computing device 104 identifies a food provider 128 with a nourishment theme such as Italian American and comparing includes determining if one or more nourishment provisions can be modified to be made gluten free to compare a nourishment intake theme 136 of gluten free. In yet another non-limiting example, comparing includes finding food provider 128 that offer nourishment provisions that are low in fermentable oligosaccharides, disaccharides, monosaccharides, and polyols (FODMAPS) to meet the needs of a nourishment intake theme 136 of low-FODMAP.

With continued reference to FIG. 5, at step 530, computing device 104 supports transmission of a nourishment provision as a function of comparing a first nourishment intake theme to a nourishment provider theme. Supporting transmission includes transmission to a remote device 108 information identifying a food provider 128 that contains a nourishment provision that compares a nourishment intake theme 136. Computing device 104 transmits information identifying a food provider 128 utilizing any network methodology as described herein. Computing device 104 may transmit information such as advertising materials that may promote and/or display one or more food provider 128 that contain nourishment provisions that compare a nourishment intake theme 136. Advertising materials include any marketing communications that may contain sponsored messages to promote a food provider 128 and/or to sell nourishment provisions offered by a food provider 128. Computing device 104 supports transmission of default food provider located within a specified location 112. A default food provider includes any of the default food provider as described above in more detail in reference to FIG. 1. For example, a default food provider may include a food provider 128 that does not have any nourishment provisions that compare a nourishment intake theme 136. In yet another non-limiting example, a default food provider may include any food provider 128 located within a specified location 112, which may have not yet been analyzed to determine if the default food provider offers nourishment provisions that compare a nourishment intake theme 136. Computing device 104 is configured to block transmission of nourishment provisions outside a nourishment intake theme 136. Blocking transmission includes not transmitting to a remote device 108, information pertaining to nourishment provisions that do not compare a nourishment intake theme 136. For instance and without limitation, computing device 104 blocks transmission of nourishment provisions that contain dairy and that cannot be modified to remove and/or modify dairy for a dairy free nourishment intake theme 136. In such an instance, computing device 104 supports transmission of nourishment provisions that do not contain dairy offered by the same food provider 128.

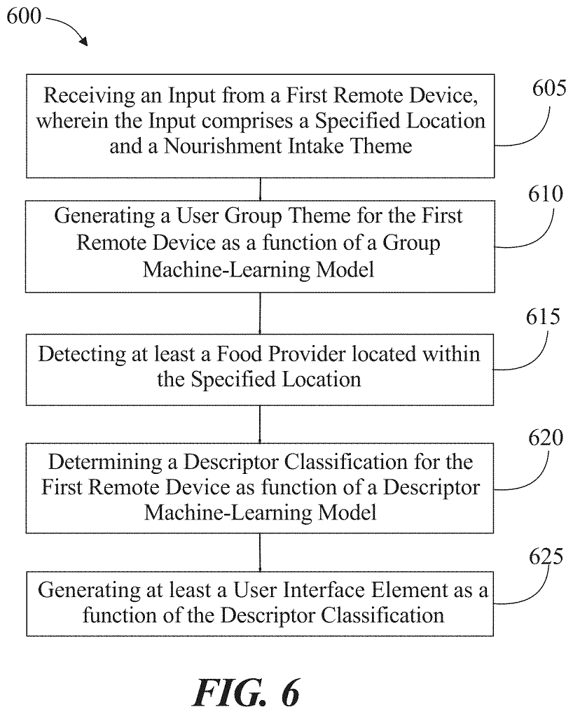

Now referring to FIG. 6, a method 600 of generating advertisements based on user group is presented. In embodiment, method 600, at step 605, includes receiving an input from first remote device 108 from a plurality of remote devices 108, wherein the input includes specified location 112 and nourishment intake theme 136. Method 600 may be consistent with disclosure with reference to FIGS. 1-4 and 7.

Still referring to FIG. 6, in an embodiment, method 600, at step 610, includes generating user group theme 204 for first remote device 108 as a function of group machine-learning model 208, wherein group machine-learning model 208 receives nourishment intake theme 136 and specified location 112 as input and outputs user group theme 204. Method 600 may be consistent with disclosure with reference to FIGS. 1-4 and 7.

Continuing to refer to FIG. 6, in some embodiments, method 600, at step 615, includes identifying at least a food provider 128 located within specified location 112. Method 600 may be consistent with disclosure with reference to FIGS. 1-4 and 7.

With continued reference to FIG. 6, at step 620, method 600, in an embodiment, includes generating descriptor classification 212 for first remote device 108 as a function of descriptor machine-learning model 216, wherein descriptor machine-learning model 216 receives at least a food provider 128 and user group theme 204 as input, and outputs descriptor classification 212. Method 600 may be consistent with disclosure with reference to FIGS. 1-4 and 7.

Still referring to FIG. 6, in some embodiments, method 600, at step 625, includes determining at least user interface element 220 as a function of the descriptor classification 212. Method 600 may be consistent with disclosure with reference to FIGS. 1-4 and 7.

Continuing to refer to FIG. 6, in an embodiment, method 600 may include transmitting at least a user interface element 220 to other remote devices. Method 600 may be consistent with disclosure with reference to FIGS. 1-4 and 7.

With continued reference to FIG. 6, method 600 may include, in an embodiment, receiving, by the computing device, food interest data from first remote device 108 and generating an interest group theme as a function of an interest machine-learning model, wherein the interest machine-learning model receives the food interest data and the specified location as input and outputs the interest group theme. In some embodiments, method 600 may further include generating a targeted advertisement theme using a targeted machine-learning model, wherein the targeted machine-learning model receives at least a food provider 128 and the interest group theme as inputs and outputs the targeted advertisement theme and determining, by the computing device, the at least an advertising material for the first remote device as a function of the targeted advertisement theme. Method 600 may be consistent with disclosure with reference to FIGS. 1-4 and 7.

Still refereeing to FIG. 6, In embodiments, method 600 may include receiving an availability datum. In further embodiments, method may include determining the at least a user interface element 220 for the first remote device 108 as a function of descriptor classification 212 and the availability datum. In an embodiment, method 600 may include receiving a promotional datum. In further embodiments, method 600 may include determining at least a user interface element 220 for first remote device 108 as a function of the advertisement theme and the promotional datum. In some embodiments, method 600 may include identifying at least a food provider 128 as a function of a bid ranking. In embodiments, method 600 may include receiving advertisement feedback. In further embodiments, method 600 may include transmitting user interface element 220 to other remote devices 108 based on the advertisement feedback. Method 600 may be consistent with disclosure with reference to FIGS. 1-4 and 7.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

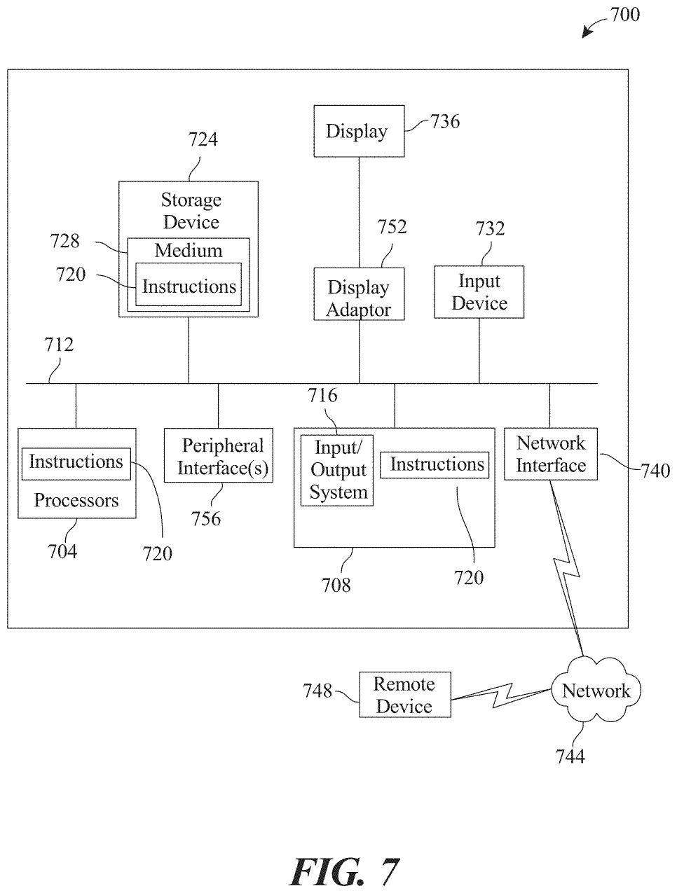

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Memory 708 may include various components (e.g., machine-readable media) including, but not limited to, a random access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for generating textual outputs based on descriptor classifications, the system comprising a computing device, the computing device designed and configured to:
receive an input from a first remote device of a plurality of remote devices, wherein the input comprises a specified location and a nourishment intake theme;
generate, as a function of a group machine-learning model, a user group theme for the first remote device, wherein the group machine-learning model receives the nourishment intake theme and the specified location as an input, and outputs the user group theme;
identify at least a food provider located within the specified location;
determine, as a function of a descriptor machine-learning model, a descriptor classification for the first remote device, wherein the advertisement machine-learning model receives the at least a food provider and the user group theme as input and outputs the descriptor classification; and
generate at least a user interface element at the first remote device as a function of the descriptor classification.

2. The system of claim 1, wherein the computing device is further configured to:
receive food interest data from the first remote device; and
generate an interest group theme as a function of an interest machine-learning model, wherein the interest machine-learning model receives the food interest data and the specified location as input and outputs the interest group theme.

3. The system of claim 2, wherein the computing device is further configured to:
determine a targeted theme using a targeted machine-learning model, wherein the targeted machine-learning model receives the at least a food provider and the interest group theme as inputs and outputs the targeted theme; and
generate the at least a user interface element at the first remote device as a function of the targeted theme.

4. The system of claim 1, wherein computing device is configured to receive an availability datum.

5. The system of claim 4, wherein the computing device is further configured to determine the at least an advertising material for the first remote device as a function of the advertisement theme and the availability datum.

6. The system of claim 1, wherein computing device is configured to receive a promotional datum.

7. The system of claim 6, wherein the computing device is further configured to generate the at least a user interface element at the first remote device as a function of the descriptor classification and the promotional datum.

8. The system of claim 1, wherein the computing device is further configured to identify the at least a food provider as a function of a bid ranking.

9. The system of claim 1, wherein the computing device is further configured to receive interface feedback.

10. The system of claim 9, wherein the computing device is further configured to transmit the at least a user interface element to other remote devices based on the interface feedback.

11. A method of generating textual outputs based on descriptor classifications, the method comprising:
receiving, by a computing device, an input from a first remote device of a plurality of remote devices, wherein the input comprises a specified location and a nourishment intake theme;
generating, by the computing device, a user group theme for the first remote device as a function of a group machine-learning model, wherein the group machine-learning model receives the nourishment intake theme and the specified location as an input, and outputs the user group theme;
identifying, by the computing device, at least a food providers located within the specified location;
determining, by the computing device, a descriptor classification for the first remote device as a function of a descriptor machine-learning model, wherein the descriptor machine-learning model receives the at least a food provider and the user group theme as input and outputs the descriptor classification; and
generating, by the computing device, at least a user interface element at the first remote device as a function of the descriptor classification.

12. The method of claim 11, wherein the method further comprises:
receiving, by the computing device, food interest data from the first remote device; and
generating, by the computing device, an interest group theme as a function of an interest machine-learning model, wherein the interest machine-learning model receives the food interest data and the specified location as input and outputs the interest group theme.

13. The method of claim 12, wherein the method further comprises:
determining, by the computing device, a targeted theme using a targeted machine-learning model, wherein the targeted machine-learning model receives the at least a food provider and the interest group theme as inputs and outputs the targeted theme; and
generating, by the computing device, the at least textual output at the first remote device as a function of the targeted theme.

14. The method of claim 11, wherein the method further comprises receiving, by the computing device, an availability datum.

15. The method of claim 14, wherein the method further comprises determining, by the computing device, the at least an advertising material for the first remote device as a function of the advertisement theme and the availability datum.

16. The method of claim 11, wherein the method further comprises receiving, by the computing device, a promotional datum.

17. The method of claim 16, wherein the method further comprises generating, by the computing device, the at least a user interface element at the first remote device as a function of the descriptor classification and the promotional datum.

18. The method of claim 11, wherein the method further comprises identifying, by the computing device, the at least a food provider as a function of a bid ranking.

19. The method of claim 11, wherein the method further comprises receiving, by the computing device, interface feedback.

20. The method of claim 19, wherein the method further comprises transmitting, by the computing device, the at least a user interface element to other remote devices based on the interface feedback.
